(19) United States
(12) Patent Application Publication
(10) Pub. No.: US 2006/0167408 A1
(43) Pub. Date: Jul. 27, 2006

(54) Single-use syringe incorporating a sliding protection sheath for the needle

(76) Inventor: Olexander Ashotovich Amatuni, Simpferopol (TR)

Correspondence Address: ARENT FOX PLLC, 1050 CONNECTICUT AVENUE, N.W., SUITE 400, WASHINGTON, DC, 20036, US

(21) Appl. No.: 10/521,363

(22) PCT Filed: Apr. 2, 2003
(86) PCT No.: PCT/TR03/00031

§ 371 Date: Dec. 6, 2005

(30) Foreign Application Priority Data
Jul. 15, 2002 (UA) 2002075820

(51) Int. Cl. A61M 5/00; A61M 5/32

(52) U.S. Cl. 604/110; 604/192

(57) ABSTRACT

The present invention relates to single-use injection syringes having means of protection from re-use and more importantly from traumatizing by a needle after injection. Application of said medical syringe allows eliminating its reuse, guaranteeing prevention from traumatizing by the distal end of the needle after injection and excluding the possibility of carry incurable or difficult to cure infection diseases. A protective sheath covering the needle of the syringe is automatically advanced to cover the distal end of said needle upon completion of its single use. A thin-walled protective sheath is further installed circumferentially onto the syringe needle without clearance and fixed on an actuator located in the barrel above a partition having freedom of lengthwise travel allowing to inject a needle with said sheath hypodermically/under the skin or intramuscular to a patient painlessly with the possibility of additional extension of tissue by said sheath, which in turn, allow to speed up the process of a absorption of injected medications.

The invention belongs to medical engineering, in particular to injection syringes of single-use having means of protection from re-use of a syringe and traumatizing by a needle after injection.

The known single-use syringes, which have device eliminating their reuse, for example by means of destruction of connection/conjunction of the cylinder piston with a rod after injection at reverse motion of a rod (see patent of Ukraine N^{o} 11186, cl. A 61 M 5/50, 1990; patent of the Russian Federation N^{o} 2061506, cl. A 61 M 5/50, 1996) or in the result of jamming the cylinder piston with a rod in the syringe barrel after injection (see patent of Ukraine N^{o} 11230, cl. A 61 M 5/50, 1993).

The known devices provide guaranteed single use but do not prevent from traumatizing by a needle after injection, and consequently do not defend from possible transfer/dissemination of infectious disease at use of a syringe.

The most close analogue on achievement of technical result is the injection syringe, having with the stated design solution common essential signs: a cylindrical barrel open from two sides, in one of which is the syringe needle with an axial channel, located inside the barrel piston with a rod and clamp on its end, and also means of protection from re-use of the syringe and prevention from traumatizing by the distal end of a needle after injection, has been adopted as prototype (see patent of the Russian Federation N^{o} 2062118, cl. A 61 M 5/50, 1996).

The drawback of the known device adopted as prototype, except the complexity of manufacturing (ten structural elements, including two threading joints), is a number of composite manipulations after extraction of a needle from a body of the patient, stipulated by the necessity of moving a rod forward to lock the connecting plates of the cylinder piston with a lug/cam, on which is installed cannula with a syringe needle, and subsequent rotary motion of a rod with the cylinder piston for unscrewing the lug with a needle from the syringe barrel and moving of a rod together with a lug and needle to the chamber, where the rod is closed by a tongue clamp.

In the basis of the invention has been the task of creation the injection syringe ensuring guaranteed single use and prevention of random traumatizing by a needle by automatic closure of its distal end at final motion of the cylinder piston in process the of making injection.

Technical outcome of the invention is the simplification of the design and engineering of the secure use of injection syringe.

The task and technical result are obtained by the fact that the injection syringe, having with the stated design solution common essential signs: a cylindrical barrel open from two sides, in one of which is the syringe needle with an axial channel, located inside the barrel piston with a rod and clamp on its end, and also means of protection from reuse of the syringe and prevention from traumatizing by the distal end of a needle after injection. Its peculiarity is that this syringe additionally contains a partition/seal, actuator and diaphragm located above the piston. Thus the injection needle is hardly mounted on the partition that has diametrically arranged windows, and the bottom part of the injection needle is installed under partition and passes/runs through the diaphragm for connection of the axial channel with the chamber of the barrel, and device protecting from re-use of a syringe and traumatizing by a needle is manufactured as thin-walled sheath, that is installed without clearance on a syringe needle and fixed on actuator located in the barrel above the partition with the ability of lengthwise travel and having an axial channel for pass of the injection needle and diametrically arranged legs for pass through windows of the partition, the legs of the actuator are supplied with wedge-shaped supports with installed above and below pivotal/bearing/supporting sites for locking actuator on the partition at open and closed positions of distal end of the syringe needle.

The indicated essential signs are indispensable and sufficient for implementation of the invention and achievement of technical result.

The invention is also peculiar by the fact that the diaphragm is executed in the form of a bushing located below funnel of domed surface passing into an axial channel, at the end of which is thin-walled partition punched at syringe assembly by the rear point of the syringe needle for connection of its channel with the barrel chamber and at the top has stepped in cross-section cylindrical cavity, thus the piston from above is supplied with the cone for going into the funnel at transfer of the actuator with the protective sheath by the diaphragm along a syringe needle and blocking/covering its distal end.

Besides the barrel of the syringe at the rear butt end/face has a cylindrical turnery, the form of which corresponds to the form of the rod clamp.

Besides the legs of the actuator from an exterior side have downwards-directional V-shaped lugs/cams, the form of which corresponds to the form of the stepped cavity of the top of the diaphragm.

These signs are referred to the optional, since they update/clarify the main signs and can be exchanged by other.

In another example of the manufacture of the syringe, the invention is peculiar by the partition which is supplied with located below fitting pipe/nipple, co-axial to the barrel, for fixation of the rear end of the injection needle, and the axial channel of the diaphragm is executed by a sheer /through pass into it of this nipple and connection of the channel of the injection needle with the barrel chamber.

The relationship of casual-resultative effect of the essential signs and achieved technical result is in the following:

1. Installation of a thin-walled clearance free protective sheath on a syringe needle, coherent with means of its lengthwise travel, allows to inject a needle with a sheath hypodermically /under the skin or intramuscular to the patient painlessly with a possibility of additional extension of tissue by a sheath, that, in its turn, allows to speed up the process of a absorption of injected drugs/medications. At this the distal end of a needle is covered by a thin sheath directly in the muscle tissue of the patient up to extraction of the needle and thus avoid the possibility of traumatizing by a needle and reuse of a syringe.

2. Location of the seal/partition with diametrically arranged windows within the barrel walls and hard-fastening of the injection needle on it with location at its tail end below, allows to create a steady position of a needle in the syringe barrel and increase safety of injection.

3. Installation of the protective sheath on the actuator installed in the barrel above the partition with the ability/possibility of lengthwise travel and having axial channel for pass of the injection needle and diametrically located legs for pass through the windows of the partition, allows to connect the actuator with the diaphragm and execute its shifting of the actuator at the final motion of the cylinder piston for guaranteed blocking of the distal end of the syringe needle in the process of injection.

4. Fulfillment on legs of the actuator of the oppositely arranged V-shaped supports with the bearing sites above and below allows to automatically close the actuator on the partition at the open and closed positions by a thin sheath of the distal end of a needle and to eliminate the possibility of its spontaneous opening.

5. Fulfillment of V-shaped supports at the ends of actuator's legs, the form of which corresponds to the form of a stepped cavity of the top part of the diaphragm, allows to prevent disconnection of the diaphragm and actuator at rarefaction in the chamber arising at fast intake of liquid by the syringe.

6. Fulfillment from butt end of the barrel the cylindrical turnery and placement behind it of a V-shaped ledge, the form of which corresponds to the form of the rod clamp and allows to clamp the rod at the end of injection to the mentioned ledge with the possibility of reliable fixation and prevention of rod's movements from the barrel in the attempt to make another injection.

7. Fulfillment of the diaphragm is executed in the form of a bushing located below funnel of domed surface passing into an axial channel, at the end of which is thin-walled partition punched at syringe assembly by the rear point of the syringe needle, and piston with the cone in the front part that allows to compact the needle in the diaphragm and increase the volume of injected liquid.

8. Fulfillment of the partition in the syringe barrel with located below co-axially to the barrel nipple/fitting pipe and fixation in it of the rear end of the syringe needle, and axial channel of the diaphragm through, allows to improve reliable fixation of the syringe needle on the partition, its centering on the axis of the barrel and improve its tight connection with the diaphragm.

Thus, the following technical result has been achieved.

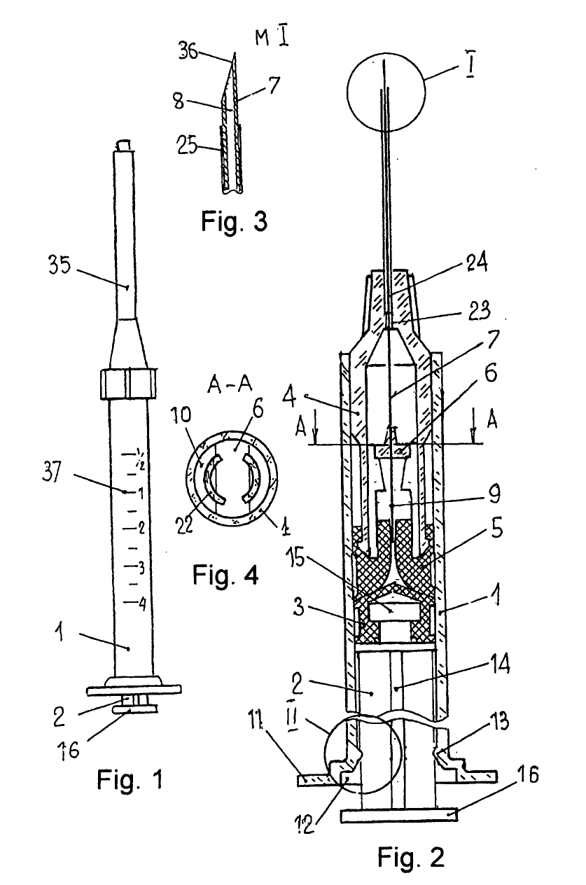

On FIG. 1 the general view of the injection syringe;

FIG. 2 same, longitudinal section of the syringe prepared for intake of liquid;

FIG. 3 place 1 on FIG. 2 distal end of the syringe needle;

FIG. 4 section A-A on FIG. 2 the installation of actuator's legs to the windows of the partition;

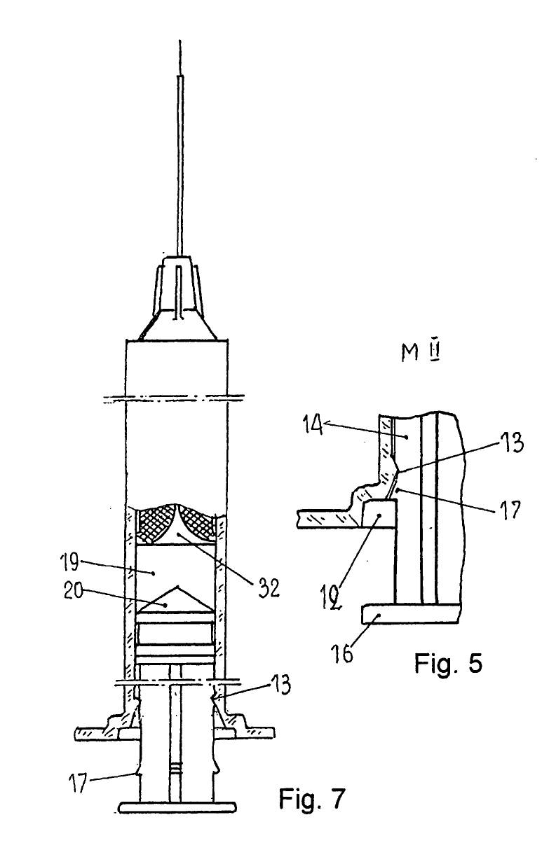

FIG. 5 place 11 on FIG. 2 V-shaped detent of a rod and cylindrical ledge in the syringe barrel;

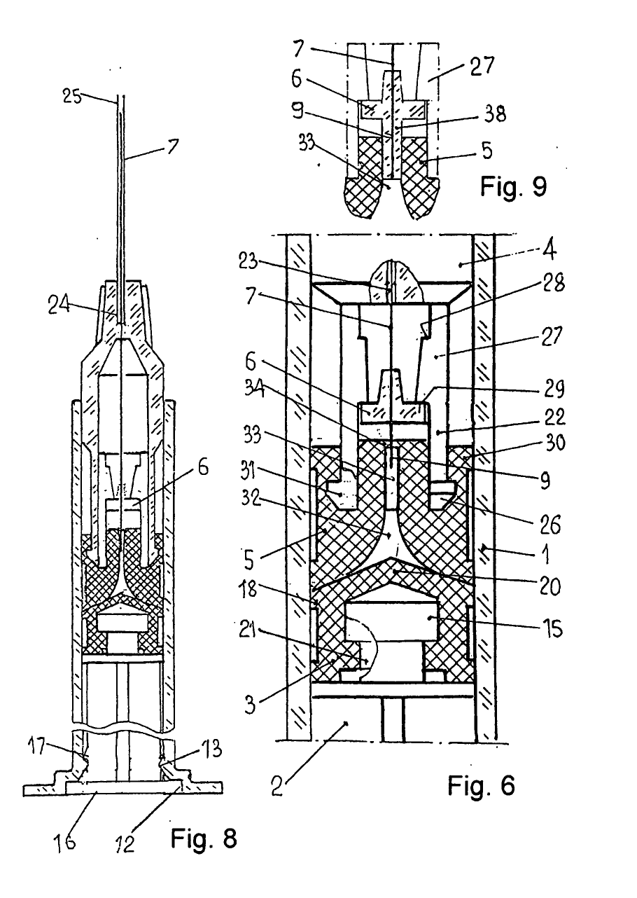

FIG. 6 part of the syringe showing the position of the actuator at completion of injection;

FIG. 7 same at intake of liquid;

FIG. 8 the longitudinal section of the syringe after use

On FIG. 9 another example of the fulfillment of connection of the syringe needle with the diaphragm is shown.

Injection syringe (FIGS. 1, 2 and 4) consists of a barrel 1 and located in it with the possibility of lengthwise travel rod 2 with the cylinder piston 3, actuator 4 and diaphragm 5, located between the actuator and cylinder piston. The barrel 1, made from the polymer material, is opened from two sides and in the middle from the front side has a partition 6 in the form of the rod, in which the syringe needle 7 is firmly fixed with an axial channel 8 and rear end, outstanding under the rod, 9. On each side of partition 6 and internal surface of the barrel 1 are created windows 10 (FIG. 4). Outside and at the rear end barrel 1 has a stop plate 11, and in its butt end-cylindrical turnery 12 and located behind it cylindrical V-shaped in cross-section ledge 13 (FIGS. 2 and 5). Rod 2 made from polymer in the form of a rod with fins of rigidity/hardness 14 and has in front stepped tip 15 for installation of the cylinder piston 3, and behind-clamp 16, proportional cylindrical turnery 12 and V-shaped detents 17 (FIG. 5 and 8), directed to the opposite side from ledge 13 of barrel 1 for fixation of rod 2 in the front extreme position, when its clamp 16 will enter flush cylindrical turnery 12 of barrel 1. The cylinder piston 3 is made from bergaflex (modified rubber) in the form of a cap with compressive cylindrical ledges 18 (FIGS. 6 and 7) on its outside surface, diameter of which exceeds internal diameter of chamber 19 of barrel 1 and created in front cone 20. Inside the cap has a stepped cavity 21, conforming to the form/shape of tip 15 for its reliable fixation on the rod 2. Actuator 4 is made from polymer material is a barrel, proportional to the internal diameter of the front end of a barrel 1 with legs 22, through axial channel 23 for pass of the injection needle 7 and concentric to it cylindrical jack/socket 24 (FIG. 8) for the installation of a thin protective sheath 25 of injection needle 7. Outside at the end of legs 22 are created directed back V-shaped ledges 26, and in the middle—oppositely arranged forward directional V-shaped supports 27 with upper 28 and lower bearing sites 29 for interaction with partition 6 in the front and back positions of protective sheath 25 of injection needle 7. Diaphragm 5 is made from bergaflex in the form of a bushing with cylindrical compressor ledges 30 at the outside surface, the size of which exceeds the diameter of chamber 19, and inside it there is stepped cavity 31, conforming to the form/shape and sizes of leg ends 22 for reliable fixation of the diaphragm on the actuator and avoidance of their disconnection at intake of liquid by the syringe. Behind diaphragm 5 is conical funnel 32 domed surface with central cylindrical channel 33, at the end of which is a thin-walled partition 34, punched at assembly of the syringe by a rear end 9 of injection needle 7 for connection of its channel 8 with chamber 19 of barrel 1 and seal it with the diaphragm. In the non-operating condition, the syringe prepared for intake of liquid, injection needle 7 is closed by cap 35, and the syringe is packaged into a protective case (not shown). In this case the distal end of injection needle 36 protrudes from protective sheath 25, and rod 2 is protruded from barrel 1 to 5 mm.

In another example of fulfillment of the syringe, fitting pipe/nipple 38 is created below partition co-axial to barrel 1, in which the rear end 9 of injection needles 7 is hard-mounted, and axial channel 33 of diaphragm 5 made through and the said fitting pipe 38 enters it for connection of the injection needle with chamber 19 of the barrel.

Unpacking the syringe and removing the cap 35 (FIG. 1), the user lowers a syringe needle into liquid and holding on barrel 1 for supporting plate 11 extends rod 2 to the required length, disconnecting diaphragm 5 and piston 3. As a result of the rarefaction in chamber 19 of barrel 1, required quantity of liquid determined on scale 37 comes to the barrel, thus compressor cylindrical ledges 18 and 30 piston 3 and diaphragm 5 provide in the chamber 19 indispensable seal, and small friction coefficient of bergaflex on polymer material, achieved with the help of lubrication by silicone, create small effort of moving piston. At injection needle 7 together with protective sheath 25 enter a body of the patient and depress clamp 16, thus the skin and muscle tissues are sequentially extended by a needle and protective sheath, that promotes acceleration of medication absorption, and the small thickness of walls of the protective sheath and practically non-clearance location of a syringe needle in it, does not augment morbid sensations of the patient. At final movement of rod 2 its clamp 16 enters flush cylindrical turnery 12 at butt end of barrel 1, and the wedge-shaped detents 17 on fins 14 close on a ring-type ledge 13, providing reliable fixation of rod 2 and impossibility of its extraction for a repeated injection. Displacing liquid from chamber 19 of barrel 1 through axial channel 8 of injection needle 7, cone 20 piston 3 enters in the conical funnel 32 of diaphragm 5 and moves/pivots actuator 4 with protective sheath 25 concerning injection needle 7 and overlaps its distal end 36 before its extraction from the body of the patient, preventing traumatizing of the medical staff. Thus supports 27 by their lower site 29 are locked on partition 6, reliably fixing the protective sheath 25 on injection needle 7 in the closed/retracted position of its distal end.

The application of the said medical syringe allows eliminating its reuse, guaranteeing prevention from traumatizing by the distal end of the needle after injection and excluding the possibility of carry incurable or difficult to cure infection diseases.

1. An injection syringe comprising a barrel 1 and a rod 2 located inside said barrel with the possibility of lengthwise travel, a needle 7 which is circumferentially covered by a thin-walled protective sheath 25, and a cylinder piston 3 in the form of a cap with compressive cylindrical ledges 18 on its outside surface, the diameter of which slightly exceeds internal diameter of chamber 19 of said barrel 1 and with a front cone 20, characterized in that the syringe further comprises,
a partition 6, which is stationary inside the barrel 1, and windows 10 on each side of said partition 6, whereby the syringe needle is firmly fixed in the central part and space is provided inside said barrel for transitional movement,
a barrel shaped actuator 4 having legs 22 and an axial channel 23 providing means for passage of said injection needle 7 and concentric to said channel, a cylindrical jack/socket 24 for installation of a thin-walled protective sheath 25 circumferential to said injection needle 7, the actuator further comprising oppositely located V-shaped ledges 26 fixed outside at the end of said legs 22 and in the middle—oppositely arranged forward directional V-shaped supports 27 with upper 28 and lower sites 29 bearing said partition 6 in the front and back positions of said protective sheath 25, whereby the actuator and the sheath gains limited transitional movement freedom on the axis of the barrel relative to both the barrel and to the needle remaining fixed on said partition 6,
a diaphragm 5, located between said actuator 4 and said cylinder piston 3, being in the form of a bushing with cylindrical compressor ledges 30 at their outside surface, the size of which slightly exceeds the diameter of chamber 19, and inside it a stepped cavity 31, conforming to the form/shape and sizes of leg ends 22 whereby the legs ends 22 of the actuator can be firmly fixed into said diaphragm and disconnection of the legs is avoided during intake stage of liquid in the syringe, and whereby the front cone 20 pushes the actuator towards the distal end of the needle when the rod is inserted completely in the barrel so that the sheath, which is accompanied by the actuator, advances towards the distal end of the needle covering its tip in full to avoid the user stick the needle tip onto any unwanted surface upon extraction of the distal end from under the skin, while said protective sheath is additionally locked in its covering position thereby guaranteeing single use of the syringe in result of the engagement of the partition on the lower bearing surfaces 29.

2. An injection syringe as set forth in claim 1 wherein the syringe comprises additional means of protection from re-use of the syringe in the form of a ring-type ledge 13 engaging on V-shaped detents 17 provided on fins 14 of the rod 2.

3. An injection syringe as set forth in claim 1, wherein said protective sheath 25 is installed without clearance on a syringe needle 7 and fixed on actuator 4 located in the barrel above the partition with the ability of lengthwise travel allowing to inject a needle with said sheath hypodermically/under the skin or intramuscular to the patient painlessly with a possibility of additional extension of tissue by said sheath, that in turn, allow to speed up the process of a absorption of injected medications.

4. An injection syringe as set forth in claim 1, wherein an external diameter of a clamp 16 is arranged to be fit and fully inserted in a cylindrical turnery 12 without clearance so that the user is eliminated to pull the inserted rod 2 out of said barrel 1 upon completion of the single use the syringe.

5. An injection syringe as set forth in claim 1, wherein said partition 6 is provided with a rectangular cross-section having substantially straight sides so that the actuator 4 is locked on the partition in the rod's 2 fully inserted position to disable the rod's rearward movement.

6. An injection syringe as set forth in claim 1, wherein the cylinder piston 3 and the diaphragm 5 are made of modified rubber material.

7. An injection syringe as set forth in claim 1, wherein the protective sheath 25 is made of transparent material.

8. An injection syringe as set forth in claim 2, wherein said protective sheath 25 is installed without clearance on a syringe needle 7 and fixed on actuator 4 located in the barrel above the partition with the ability of lengthwise travel allowing to inject a needle with said sheath hypodermically/under the skin or intramuscular to the patient painlessly with a possibility of additional extension of tissue by said sheath, that in turn, allow to speed up the process of a absorption of injected medications.
